(19) United States
(12) Patent Application Publication
(10) Pub. No.: US 2024/0395379 A1
(43) Pub. Date: Nov. 28, 2024

(54) Healthcare System

(71) Applicant: YAKOV ISRAEL REANI, BNEI BRAK (IL)

(72) Inventor: YAKOV ISRAEL REANI, BNEI BRAK (IL)

(21) Appl. No.: 18/323,063

(22) Filed: May 24, 2023

(51) Int. Cl. G16H 15/00; G06F 40/40; G06Q 30/0601; G16H 10/60; G16H 20/10; G16H 40/20; G16H 50/20

(52) U.S. Cl. CPC G16H 15/00; G06F 40/40; G06Q 30/0613; G06Q 30/0639; G16H 10/60; G16H 20/10; G16H 40/20; G16H 50/20

(57) ABSTRACT

The present invention is a novel healthcare system that comprises automatic summarization technology that uses natural language processing (NLP) algorithms to extract the most important information from a given text or speech input. In doctor-patient meetings, the system summarizes the key points and action items discussed, saving valuable clinic time and making it easier to remember and act on the most critical information. According to an embodiment of the invention, the system automatically generates a prescription for the patient, schedules follow-up meetings, sets appointments with other medical personnel, and aids in hiring/purchasing required medical equipment according to the generated prescription.

### FIELD OF THE INVENTION

The present invention relates to the field of healthcare systems, specifically to a novel method and system for automating the process of summarizing medical appointments, generating accurate prescriptions, and facilitating the purchase of associated products for patients. This invention aims to improve the overall efficiency and effectiveness of healthcare delivery by reducing the time and effort required by doctors and patients during medical appointments. The invention is particularly useful for enhancing the accuracy and speed of prescription generation and aiding patients in obtaining the prescribed medication promptly and conveniently. The field of the invention encompasses various aspects of healthcare, including medical record-keeping, prescription generation, and pharmaceutical supply chain management. The present invention has the potential to significantly enhance the quality of care provided to patients and streamline the administrative processes associated with healthcare delivery.

### BACKGROUND OF THE INVENTION

Medical appointments are a crucial aspect of healthcare, but they come with various challenges and limitations. One significant challenge doctors face during appointments is the documentation process, which can be lengthy and time-consuming. Doctors need to record accurate and complete medical history, examination findings, diagnosis, and treatment plans, which can take up valuable time that could be spent with patients. Moreover, limited appointment time and language or cultural barriers can hinder effective communication between doctors and patients, leading to miscommunication and confusion.

Additionally, after the appointment, patients need to go to a pharmacy to purchase their prescribed medication, which can involve several steps, such as choosing a pharmacy, presenting the prescription, waiting for the medication to be filled, paying for the medication, and taking it as directed. However, there can be potential drawbacks, such as longer wait times due to a high volume of prescriptions or the medication not being available in stock.

To address these challenges and drawbacks, there is a need for enhanced tools that can save time and reduce costs for patients, as well as improve the efficiency of the overall healthcare system.

It is an object of the present invention to provide a healthcare system capable of streamlining the documentation process during medical appointments, reducing the time doctors spend on documentation and enabling them to spend more time interacting with patients.

It is an object of the present invention to provide a healthcare system capable of improving communication between doctors and patients by providing tools that help overcome language and cultural barriers.

It is yet another object of the present invention to provide patients with access to accurate and reliable medical information that they can understand, reducing miscommunication and confusion.

It is still another object of the present invention to enable patients to purchase their prescribed medication more efficiently, e.g., by providing them with access to online pharmacies, home delivery services, or other innovative solutions.

It is yet another object of the present invention to simplify the process of scheduling appointments with other medical professionals, such as specialists or therapists, by providing patients with access to automatic services such as online booking system.

Other objects and advantages of the invention will become apparent as the description proceeds.

### SUMMARY OF THE INVENTION

A method for summarizing a healthcare appointment between a healthcare provider and a patient, comprising:
a) recording an audio of the healthcare appointment;
b) processing the audio to generate a text transcript of the healthcare appointment;
c) cleaning up the text transcript to remove any irrelevant conversation;
d) analyzing the text transcript using natural language processing techniques to identify key topics and themes discussed in the healthcare appointment;
e) using machine learning algorithms to determine the most important parts of the healthcare appointment based on a predetermined criteria;
f) generating a summary of the healthcare appointment based on the identified key topics and themes and the most important parts determined by the machine learning algorithms; and
g) displaying the summary to a user through a user interface.

In one aspect the method further comprising:
a) identifying medications, procedures, or other interventions recommended by the healthcare provider during the healthcare appointment; and
b) generating a prescription based on the identified medications, procedures, or other interventions.

In one aspect the method further comprising:
a) summarizing any recommendations made by the healthcare provider for future appointments;
b) providing reminders for follow-up appointments; and
c) automatically scheduling follow-up appointments based on the recommendations made by the healthcare provider.

In one aspect the method further comprising providing recommendations or guidance to medical-related services, such as where to physically obtain medications with the generated prescription or rent medical equipment.

In one aspect the prescription is generated based on the identified medications, procedures, or other interventions, and provides the prescription to a pharmacy or medical supplier for fulfillment.

In one aspect the method automatically schedules follow-up appointments with the healthcare provider, and sends reminders to the patient through the user interface or other communication means.

In one aspect the method provides the patient with a list of recommended medical suppliers or pharmacies based on their geographic location and insurance coverage.

In yet another aspect, the invention relates to a method for assisting patients to purchase medication or rent medical equipment, comprising:
a) Obtaining real-time information about the availability of medical related products at nearby places of business, waiting time from queuing system associated with each place of business, and navigation data to each place of business;
b) Combining the obtained information with the generated prescription to provide a list of recommended places of business to purchase the medicines that appear in the prescription;
c) Sorting the list of recommended places according to the quickest total time that it may take to complete the purchase, by considering the predicted waiting time, distance to the place of business, and the navigation data; and
d) Allowing the user to access the list of recommended places through a dedicated application associated with the healthcare system, and to complete the purchase from a place of business in the most effective way, in terms of waiting time, distance, and other available navigation data.

In one aspect the method further comprising:
a) Integrating with APIs provided by various third-party services to retrieve real-time data related to the availability of medical products, waiting times, and navigation data in accordance with the places of business;
b) Integrating with the queuing system of each place of business to obtain waiting time data;
c) Integrating with geolocation services that identify the patient's location and provide navigation data to the recommended places of business; and
d) Integrating with the healthcare system or with electronic health record (EHR) systems to obtain prescription data and other relevant medical data.

In one aspect the method further comprising integrating with a payment gateway that allows patients to make payments for their purchases using the application itself.

In one aspect the method further comprising employing a notification system to notify patients when their orders are ready for pickup or delivery.

In yet another aspect, the invention relates to a system for automatically summarizing a healthcare appointment between a healthcare provider and a patient, comprising:
a) audio input hardware for capturing audio from the appointment;
b) speech recognition software for processing the audio and generating text transcripts;
c) natural language processing (NLP) techniques for analyzing the text and identifying key topics and themes discussed in the appointment;
d) machine learning algorithms for identifying the most important parts of the appointment based on an organization's requirements;
e) a summary generation module for generating a summary of the appointment based on the results of the natural language processing and machine learning algorithms; and
f) a user interface for displaying the generated summary to the user, which includes features such as search, filter, and sharing.

In one aspect the system further comprising an appointment management module for providing reminders for follow-up appointments and automatically scheduling appointments or providing recommendations or guidance to medical-related services.

In one aspect the system further comprising a subscription generation module for automatically generating a prescription from the summary.

In another aspect, the invention relates to a system for assisting patients to purchase medication or rent medical equipment, comprising:
a) A processor configured to obtain real-time information about the availability of medical related products at nearby places of business, waiting time from queuing system associated with each place of business, and navigation data to each place of business;
b) A recommendation engine configured to combine the obtained information with the generated prescription (or other data from the summary) to provide a list of recommended places of business to purchase the medicines that appear in the prescription (or rent/buy medical equipment), wherein the recommendation engine further configured to sort the list of recommended places according to total time that it may take to complete the purchase, by considering the predicted waiting time, distance to the place of business, and other available navigation data; and
c) A dedicated application associated with the healthcare system, allowing the user to access the list of recommended places and complete the purchase in the most effective way, in terms of waiting time, distance, and other available navigation data.

In one aspect the system further comprising:
a) An interface module configured to integrate with APIs provided by various third-party services to retrieve real-time data related to the availability of medical products, waiting times, and navigation data;
b) An interface module configured to integrate with the queuing system of each place of business to obtain waiting time data;
c) An interface module configured to integrate with a payment gateway that allows patients to make payments for their purchases using the application itself;
d) An interface module configured to integrate with geolocation services that identify the patient's location and provide navigation data to the recommended places of business; and
e) An interface module configured to integrate with electronic health record (EHR) systems to obtain prescription data and other relevant medical data.

In one aspect the system further comprising a notification system configured to notify patients when their orders are ready for pickup or delivery.

In one aspect, the system further comprises a search/recommendation engine configured to receive a query for a desired product from the generated prescription and retrieve purchase-related data associated with the product from a plurality of components and services, wherein the purchase-related data includes availability, prices, distance, waiting times, and delivery options; a ranking algorithm configured to analyze the purchase-related data and generate a ranked list of search results based on a weighting of the purchase-related data, including predicted waiting times, geolocation with respect to the user's current location, and prices; and a user interface configured to display the ranked list of search results to the user, wherein the user can select a desired product from the search results and complete a purchase or rental transaction through the system.

### BRIEF DESCRIPTION OF THE DRAWINGS

In the drawings:

FIG. 1 is a block diagram generally illustrating a healthcare system, according to an embodiment of the present invention;

FIG. 2 is a flowchart generally illustrating the method of the invention, according to which the healthcare system may work;

FIG. 3 schematically illustrates a block diagram of a part of the system for accessing medical products and services, according to an embodiment of the invention;

FIG. 4 is a block diagram illustrating of a system for facilitating the purchase of medication or rental of medical equipment, according to an embodiment of the invention; and

FIG. 5 is a flowchart generally illustrating the method of facilitating the purchase of medication or rental of medical equipment, according to an embodiment of the invention.

### DETAILED DESCRIPTION OF THE INVENTION

The present invention is a novel healthcare system that comprises automatic summarization technology that uses natural language processing (NLP) algorithms to extract the most important information from a given text or speech input. In doctor-patient meetings, the system summarizes the key points and action items discussed, saving valuable clinic time and making it easier to remember and act on the most critical information. According to an embodiment of the invention, the action items may include: automatically generating prescription for the patient, scheduling follow-up meetings (may require patient approval for dates and time), setting appointments with other medical personnel, aiding in hiring/purchasing required medical equipment, etc.

According to one embodiment of the invention, summarization involves identifying and extracting the most critical sentences or phrases from the original text or speech input. According to another embodiment of the invention, summarization involves generating a new summary that may not be present in the original input.

According to an embodiment of the invention, utilizing a summarization that is based on identifying and extracting the most critical sentences or phrases from the speech input may involve the following procedures:
1. Speech-to-Text (STT): applying speech recognition technology to transcribe the meeting audio into text, e.g., by using tools such as Otter.ai and Fireflies.ai, by using real-time speech recognition technology to display a live transcript during the meeting, which can then be used for summarization, or any other suitable STT technology.
2. Text preprocessing: The input text is preprocessed to remove stop words (words that are commonly used but do not add much meaning, such as “the” or “and”) and perform stemming (reducing words to their root form, such as “running” to “run”).
3. Sentence extraction: The input text is segmented into sentences, and a scoring algorithm is used to identify the most important sentences based on criteria such as keyword frequency, sentence length, and position within the document.
4. Summary generation: The most important sentences are combined to form a summary that captures the key points of the meeting.

### Software and Hardware Components

Implementing an automatic summarization system for meetings requires a combination of software and hardware components. According to an embodiment of the invention, the system may include the following components:
1. Speech recognition software: To transcribe the audio of the meeting into text, the system uses speech recognition software that is capable of accurately converting speech into text.
2. Natural language processing software: To analyze and summarize the text generated by the speech recognition software, the system uses natural language processing (NLP) software that is configured to identify key phrases, topics, and themes associated with healthcare and medical terminology. For example, there are several Natural Language Processing (NLP) engines and solutions available that can be used to implement healthcare system of the present invention, such as Google Cloud Natural Language, Microsoft Azure Text Analytics, IBM Watson Natural Language Understanding, Amazon Comprehend, Spacy, and the like. Such NLP tools can be used to analyze meeting transcripts and identify key topics and themes.
3. Machine learning models: Machine learning models are used to train the system to recognize the most important parts of the meeting based on the medical and health needs, such as prescriptions, medical and health conditions, medications, medical services, etc.
4. Data storage and retrieval: The system uses a database configured to store and retrieve data quickly to maintain the historical data and previous meeting summaries.
5. Server infrastructure: The system uses server infrastructure to support the processing and storage of data. This may include cloud-based services or dedicated servers.
6. Hardware for audio capture: The system uses hardware to capture the audio from the meeting, such as microphones or other audio input devices (as input to an STT module).
7. Human-in-the-loop component: Depending on the requirements and complexity of the system, it may require the doctor (or other professional healthcare personnel) to verify and edit the generated summaries.
8. User interface: The summarization system uses a user interface to interact with the system, such as a web application or a mobile app.

According to an embodiment of the invention, implementing a meeting summarization by the healthcare system requires a combination of software and hardware components, including speech recognition software, natural language processing software, machine learning models, data storage and retrieval, server infrastructure, audio capture hardware, and a user interface. The selection and integration of these components depend on the specific requirements and goals of the healthcare organization.

Reference will now be made to several embodiments of the present invention, examples of which are illustrated in the accompanying figures for purposes of illustration only. One skilled in the art will readily recognize from the following description that alternative embodiments of the structures and methods illustrated herein may be employed without departing from the principles of the claimed invention.

The following discussion is intended to provide a brief, general description of a suitable computing environment in which the invention may be implemented. While the invention will be described in the general context of program modules or codes that execute in conjunction with an application program that runs on an operating system on a computer system, those skilled in the art will recognize that the invention may also be implemented in combination with other program modules. The functions described herein may be performed by executable code and instructions stored in a computer-readable medium and running on one or more processor-based systems. Embodiments of the invention may be implemented as a computer process, e.g., a computer system that encodes a computer program of instructions for executing the computer process.

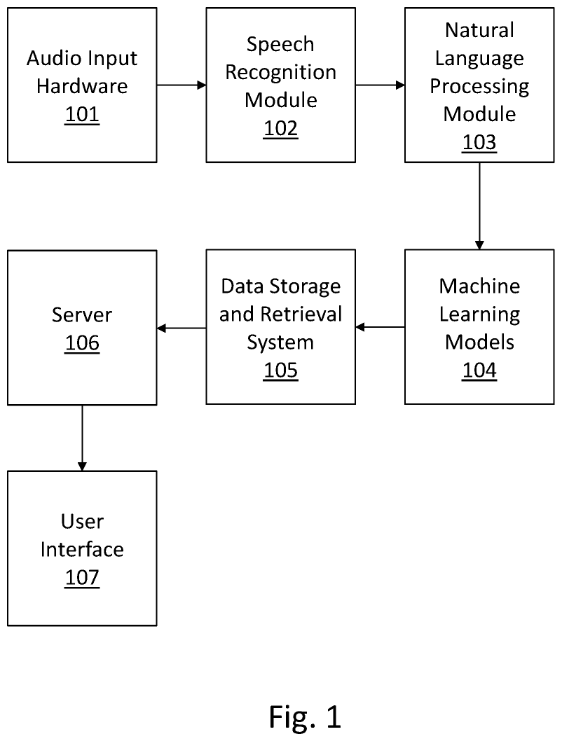

FIG. 1 is a block diagram of an automatic summarization system for meetings, according to an embodiment of the invention. The system includes at least some of the following software and hardware components:

The block diagram shows the components involved in an automatic meeting summarization system, starting with the audio input hardware 101, which is then processed by speech recognition software 102 to convert the audio into text. The text is then analyzed using natural language processing software 103, which identifies the key phrases, topics, and themes discussed in the meeting. Machine learning models 104 are used to recognize the most important parts of the meeting, based on the organization's requirements. The data is stored and retrieved using a data storage system 105, and a server 106 is used to support the processing and storage of data. Finally, the user interface 107 is used to interact with the system, allowing the user to access and review the generated summaries.

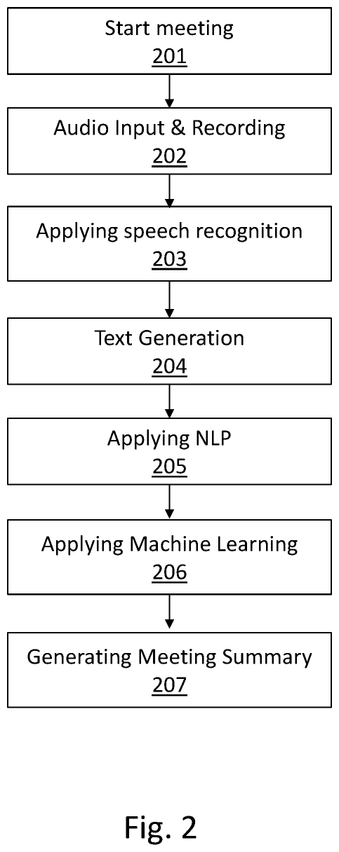

FIG. 2 is a flowchart generally illustrating the method of the invention, according to which the healthcare system may work.

The flowchart illustrates the high-level steps involved in an automatic meeting summarization system for healthcare: The system starts by recording the meeting audio between a doctor and a patient. The audio is then processed using speech recognition software to generate text. The text is then cleaned up and processed using natural language processing techniques to identify the key topics and themes discussed in the meeting. Machine learning algorithms are used to identify the most important parts of the meeting based on the medical needs of the patient. The system then generates a summary of the meeting, which is displayed to the doctor and patient using a user interface. Finally, the meeting ends, and the system is ready for the next appointment.

A detailed description of each step in the automatic meeting summarization system for the healthcare flowchart:
1. Start Meeting (201): This step involves initializing the system and starting the meeting recording process between the doctor and patient.
2. Audio Input & Recording (202): The audio input hardware captures the audio from the meeting and records it for processing.
3. Speech Recognition Software (203): The recorded audio is processed by speech recognition software to generate text transcripts of the meeting.
4. Text Generation & Cleanup (204): The text transcripts are cleaned up to remove any background noise, filler words, or any irrelevant conversation that is not related to the patient's medical needs.
5. Natural Language Processing (205): The text is analyzed by applying natural language processing techniques to identify the key topics, themes, and phrases discussed in the meeting. This involves breaking down the text into its constituent parts, such as sentences and words, and analyzing their meaning, context, and relationships to identify the most relevant medical information.
6. Machine Learning Algorithms (206): Machine learning models are applied to analyze the processed text to determine the most important parts of the meeting based on the medical needs of the patient. The models use advanced algorithms to analyze the text and identify the most significant portions of the conversation related to the patient's medical condition.
7. Generate Meeting Summary (207): The system generates a summary of the meeting based on the results of the natural language processing and machine learning algorithms. The summary is typically a condensed version of the meeting that includes the most important medical points and topics discussed between the doctor and patient. At this stage, the meeting concluds and the system stops recording and processing the audio.

According to an embodiment of the invention, the meeting summary is displayed to the doctor and patient through a user interface (e.g., see user interface 107 in FIG. 1) that allows them to access and review the generated summaries. The interface can be web-based or standalone application-based, and may include features such as search, filter, and sharing.

By automating the meeting summarization process for healthcare, the system can help to save time and increase productivity by providing a condensed and accurate summary of the meeting that can be easily accessed and reviewed by both the doctor and patient. This can help improve the quality of care and patient outcomes by ensuring that all relevant medical information is captured and communicated effectively.

In addition to the main steps outlined in the automatic meeting summarization system flowchart, the healthcare system may include the following aspects:
1. Patient History: The system can summarize the patient's medical history, including previous illnesses, medications, and allergies, as well as any relevant family medical history.
2. Symptoms and Diagnoses: The system can summarize the symptoms that the patient is experiencing and any potential diagnoses that the doctor has made based on the information provided.
3. Treatment Plan: The system can summarize the treatment plan recommended by the doctor, including medications, procedures, or other interventions. The system can also track the patient's adherence to the treatment plan. For example, one way the system can track the patient's adherence to the treatment plan is by integrating with electronic health records (EHRs) to access information on the patient's prescription history, lab results, and other medical data. The system can also utilize patient-reported outcomes and surveys to track their progress and any side effects they may be experiencing. Additionally, the system can use automated notifications and reminders to prompt the patient to take their medication or follow up with their doctor. The system can also provide educational resources and personalized recommendations to help the patient manage their condition and improve adherence to the treatment plan. By collecting and analyzing this data, the system can provide the doctor with valuable insights into the patient's progress and make any necessary adjustments to the treatment plan.
4. Follow-up Appointments and action Items: The system can provide reminders for follow-up appointments and summarize any recommendations made by the doctor for future appointments. According to some embodiments of the invention, the system can use machine learning algorithms and natural language processing techniques to identify and extract relevant information from the meeting summary, such as recommended follow-up appointments or referrals to other medical services. Based on this information, the system can automatically schedule follow-up appointments for the patient and send reminders through email, SMS, or a mobile app. Additionally, the system can provide recommendations or guidance to medical-related services based on the doctor's recommendations. For example, if the doctor recommends a specific type of medical equipment, the system can provide information on where the patient can rent or purchase the equipment. If the doctor prescribes medication, the system can suggest nearby pharmacies where the patient can fill the prescription.

Examples of outcomes (including action items) from a meeting between a doctor and a patient that can be generated by the healthcare summarization system are:
1. Prescription Refill: The system can generate a summary of the appointment that includes a reminder for the patient to refill their prescription and any changes to the dosage or instructions for taking the medication.
2. Specialist Referral: If the patient requires specialized care, the system can summarize the referral to a specialist and provide information on how to make an appointment.
3. Lifestyle Changes: The system can summarize the doctor's recommendations for lifestyle changes, such as dietary changes, exercise, or stress reduction techniques.
4. Follow-up Tests: If the patient requires follow-up tests or lab work, the system can summarize the doctor's recommendations and provide reminders for scheduling the tests.
5. Health Education: The system can summarize any health education provided by the doctor, such as information on managing chronic conditions or preventing illness, and provide resources for further reading or education.

This summary provides a concise overview of the key topics discussed during the meeting and highlights the main points of agreement and areas of concern. By using an automatic meeting summarization system, the doctor and patient can quickly review the key takeaways from the meeting without having to sift through a lengthy transcript. This can save time and improve the efficiency of the meeting process.

The following is detailed information and examples about the machine learning models and their possible usages in the process.

Machine learning models play a crucial role in the automatic summarization of meetings as they help in identifying the most relevant information from a large amount of unstructured data. There are several machine learning models that can be used by the healthcare system, in particular in the process of automatic meeting summarization. The choice of model will depend on factors such as the size and complexity of the meeting transcripts, the quality of the training data, and the desired output of the summarization system. Here are some examples of possible models:
Topic modeling—a type of unsupervised learning technique that can be used to identify the main topics and themes present in a large corpus of text data. It can be used to analyze meeting transcripts and identify the most important topics discussed during the meeting;
Text classification—a supervised learning technique that can be used to classify text data into predefined categories. It can be used to classify meeting transcripts into different categories based on the topics discussed, such as project status updates, budget and expenses, timeline and deadlines, etc.
Sequence-to-sequence models—a type of deep learning technique that can be used to generate summaries from a large corpus of text data. These models can be trained on a large dataset of meeting transcripts and can be used to generate summaries of new meetings automatically.

### Examples about the Action Items and Possible Ways to Fulfill Them

Action items are specific tasks or responsibilities assigned to patients or professional medical personnel during a meeting. These tasks are important to ensure that the decisions and discussions that took place during the meeting are followed through and implemented effectively. Here are some possible ways to fulfill action items: automatically generating prescription for the patient, scheduling follow-up meetings (may require patient approval for dates and time), setting appointments with other medical personnel, aiding in hiring/purchasing required medical equipment, suggesting best ways to purchase the prescribed medications, etc.

According to an embodiment of the invention, the system comprises employing an action item for a further automation process, such as purchasing a medication or renting/purchasing medical equipment according to the prescription (or according to the doctor's recommendation).

According to an embodiment of the invention, the system assists the patient to effectively purchase medication or rent required medical equipment, by obtaining real-time information about the following:
Availability and prices of medical related products (in stock items data) at nearby places of business (e.g., pharmacies, drugstores, medical service stores, etc.);
Availability of a delivery option and timing;
Waiting times derived from a queue management system associated with each place of business; and
Navigation data to each place of business.

The system considers the above information and combines it with the generated prescription (or other data from the summary) to provide a list of recommended places of business to purchase the medicines that appear in the prescription (or rent/buy medical equipment) in the most effective way for the user. For example, the user may sort the list of recommended places according to the quickest total time that it may take to complete the purchase (i.e., by considering the predicted waiting time, distance to the place of business and other available navigation data, availability, etc.).

By using a dedicated applications associated with the healthcare system, the patient or other user on the patient behalf, is able to complete the purchase in most effective way, in terms of waiting time, distance, etc.

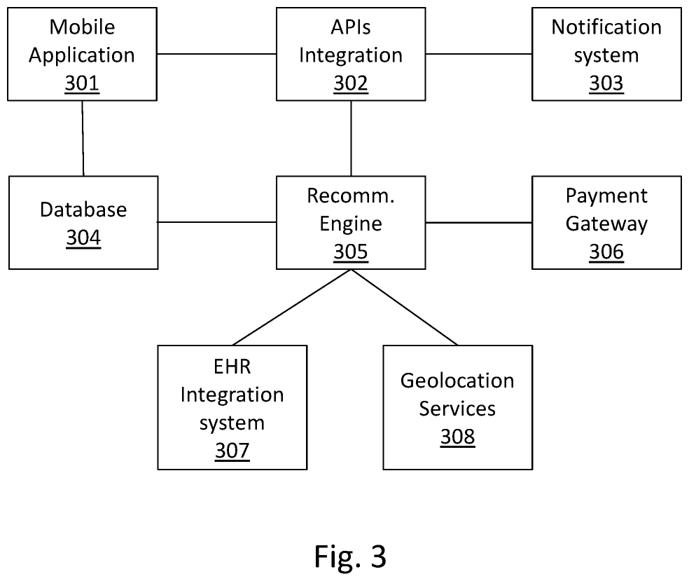

According to an embodiment of the invention, to implement the system described above, several components and services are required. FIG. 3 schematically illustrates a block diagram of the system's components and services for accessing medical products and services, according to an embodiment of the invention. As shown in this figure, the components and services can include:
1. Mobile application (301): A dedicated mobile application can be used by patients (as well as other users, such as medical staff) to access the system. For example, the application can be compatible with iOS and Android devices and can provide a user-friendly interface to access the features of the system.
2. Database (304): A database can store real-time information about the availability and prices of medical products at nearby places of business, waiting times, navigation data, and other related information. This data can be updated frequently to ensure accuracy.
3. API integration (302): The system can be integrated with APIs provided by various third-party services to retrieve real-time data related to the availability and prices of medical products, waiting times (e.g., based on inline data derived from queuing management system), and navigation data (e.g., Waze, Google Maps, etc.). Integration with the queuing management system of each place of business can also be established to calculate predicted waiting time in each place of business.
4. Recommendation engine (305): The system can use a recommendation engine to provide a list of recommended places of business to purchase the medicines that appear in the prescription or to rent/buy medical equipment. The engine can use machine learning algorithms to sort the recommended places of business based on factors such as predicted waiting time, distance to the place of business, availability, etc.
5. Payment gateway (306): A payment gateway can be integrated into the system to allow patients to make payments for their purchases using the application itself.
6. Notification system (303): A notification system can be set up to notify patients when their orders are ready for pickup or delivery.
7. Integration with EHR systems (307): The system can be integrated with electronic health record (EHR) systems to obtain prescription data and other relevant medical data.
8. Geolocation services (308): Geolocation services can be used to identify the patient's location and provide navigation data to the recommended places of business.

According to some embodiments of the invention, the system may further comprise an analytics dashboard to monitor the usage of the system, track the performance of the recommendation engine, and identify areas for improvement.

According to an embodiment of the invention, the system may work as follows:
Receive prescription data from the generated prescription and/or from the EHR system (307);
Use recommendation engine (305) to provide list of recommended places of business, to calculate predicted waiting times for each recommended place of business and to sort recommended places of business based on predicted waiting times, distance, availability, etc.;
Display sorted list to user in mobile application (301);
Allow user to select desired place of business and make payment through payment gateway (306); and
Notify user when order is ready for pickup or delivery, e.g., via notification system 303.

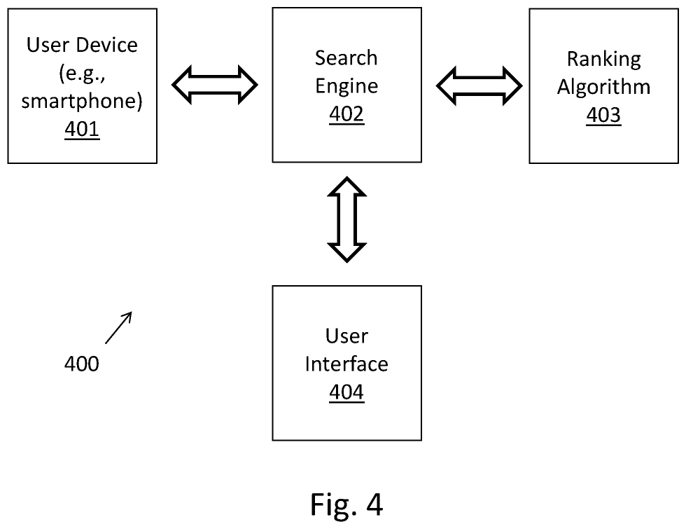

The above components and services can work together to provide an effective system that assists patients in purchasing medication or renting medical equipment in the most efficient and timely way possible. The aforementioned components and services can operate in tandem to create a robust system that facilitates patients in the seamless procurement of medicine or rental of medical equipment with optimal efficiency and timeliness. FIG. 4 is a block diagram illustrating of a system 400 for facilitating the purchase of medication or rental of medical equipment, according to an embodiment of the invention. System 400 comprises a search/recommendation engine 402, which leverages various components and services, such as availability, prices, distance, waiting times, and delivery options, to gather relevant purchase-related data for the desired product (e.g., from a prescription that was previously generated during or at the end of a doctor-patient meeting). Search engine 402 then employs a ranking algorithm 403 that factors in various purchase-related data points, including projected waiting times, geolocation with respect to the user's current location, and prices, to sort the search results and present the most relevant and useful options to the user via a user interface 404. For example, the user interface can be part of mobile application that runs on user device 401, such as a smartphone or other suitable mobile device.

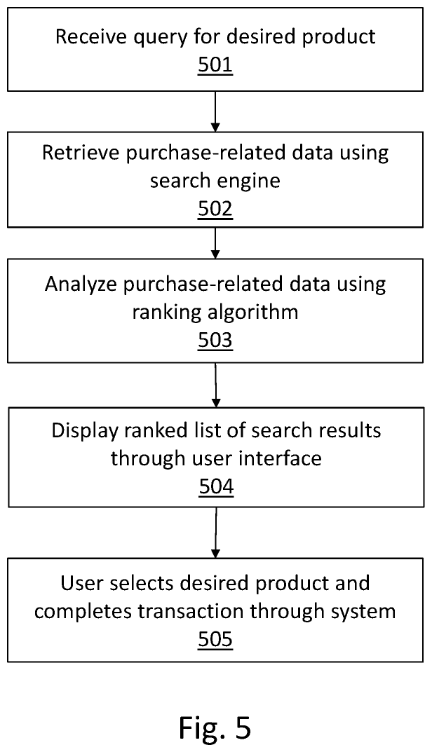

Referring now to FIG. 5, according to an embodiment of the invention, for facilitating the purchase of medication or rental of medical equipment, the system may involve the following procedures:
receiving a user's query for a desired product (step 501);
retrieving purchase-related data associated with the product from a plurality of components and services (step 502), wherein the purchase-related data includes availability, prices, distance, waiting times, and delivery options, using search/recommendation engine 402;
analyzing the purchase-related data using ranking algorithm 403 to generate a ranked list of search results based on a weighting of the purchase-related data, including predicted waiting times, geolocation with respect to the user's current location, and prices (step 503); and
displaying the ranked list of search results to the user through user interface 404 (step 504), wherein the user can select a desired product from the search results and complete a purchase or rental transaction through the system (step 505).

According to an embodiment of the invention, the search/recommendation engine may be implemented using a combination of different search techniques, such as keyword-based search, faceted search, and natural language processing. To retrieve purchase-related data associated with the desired product, the search/recommendation engine may use APIs from various components and services, such as online pharmacies, medical equipment rental providers, and logistics companies. The search/recommendation engine may also leverage user data, such as search history, purchase history, and location data, to provide more personalized and relevant recommended purchase results to the user.

According to an embodiment of the invention, the ranking algorithm may use a combination of different data points to rank the search results, such as availability, prices, distance, waiting times, delivery options, and user ratings. To calculate predicted waiting times, the ranking algorithm may use historical data of waiting times in each place of business, as well as real-time data on product availability and order volume. To calculate geolocation with respect to the user's current location, the ranking algorithm may use GPS data from the user's device, as well as IP address and Wi-Fi triangulation. In some embodiments, the ranking algorithm may also use machine learning techniques, such as neural networks or decision trees, to learn from user feedback and continuously improve the ranking of search results over time.

As will be appreciated by the skilled person, the arrangement described in the figures results in a system capable of automatically summarizing an appointment between a doctor and a patient and, in addition, providing the patient recommendations for the needed medicines and/or medical equipment.

All the above will be better understood through the following illustrative and non-limitative examples.

The terms, “for example”, “e.g.”, “optionally”, as used herein, are intended to be used to introduce non-limiting examples. While certain references are made to certain example system components or services, other components and services can be used as well and/or the example components can be combined into fewer components and/or divided into further components.

All the above descriptions and examples have been given for the purpose of illustration and are not intended to limit the invention in any way. Many different mechanisms, methods of summarizing, electronic and logical elements can be employed, all without exceeding the scope of the invention.

1. A method for summarizing a healthcare appointment between a healthcare provider and a patient, comprising:
a) recording an audio of the healthcare appointment;
b) processing the audio to generate a text transcript of the healthcare appointment;
c) cleaning up the text transcript to remove any irrelevant conversation;
d) analyzing the text transcript using natural language processing techniques to identify key topics and themes discussed in the healthcare appointment;
e) using machine learning algorithms to determine the most important parts of the healthcare appointment based on a predetermined criteria;
f) generating a summary of the healthcare appointment based on the identified key topics and themes and the most important parts determined by the machine learning algorithms; and
g) displaying the summary to a user through a user interface.

2. The method of claim 1, further comprising:
a) identifying medications, procedures, or other interventions recommended by the healthcare provider during the healthcare appointment; and
b) generating a prescription based on the identified medications, procedures, or other interventions.

3. The method of claim 1, further comprising:
a) summarizing any recommendations made by the healthcare provider for future appointments;
b) providing reminders for follow-up appointments; and
c) automatically scheduling follow-up appointments based on the recommendations made by the healthcare provider.

4. The method of claim 2, further comprising providing recommendations or guidance to medical-related services.

5. The method of claim 2, wherein the prescription is generated based on the identified medications, procedures, or other interventions, and provides the prescription to a pharmacy or medical supplier for fulfillment.

6. The method of claim 3, wherein the system automatically schedules follow-up appointments with the healthcare provider, and sends reminders to the patient through the user interface or other communication means.

7. The method of claim 5, wherein the system provides the patient with a list of recommended medical suppliers or pharmacies based on geographic location and insurance coverage.

8. A method for assisting patients to purchase medication or rent medical equipment, comprising:
a) obtaining real-time information about the availability of medical related products at nearby places of business, waiting time from queuing system associated with each place of business, and navigation data to each place of business;
b) combining the obtained information with the generated prescription to provide a list of recommended places of business to purchase the medicines that appear in the prescription;
c) sorting the list of recommended places according to the quickest total time that it may take to complete the purchase, by considering the predicted waiting time, distance to the place of business, pricing, delivery options and the navigation data; and
d) allowing the user to access the list of recommended places through a dedicated application associated with the healthcare system, and to complete the purchase from a place of business in the most effective way, in terms of waiting time, distance, and other available navigation data.

9. The method of claim 8, further comprising:
a) integrating with APIs provided by various third-party services to retrieve real-time data related to the availability of medical products, waiting times, and navigation data in accordance with the places of business;
b) integrating with the queuing system of each place of business to obtain waiting time data;
c) integrating with geolocation services that identify the patient's location and provide navigation data to the recommended places of business; and
d) integrating with the healthcare system or with electronic health record (EHR) systems to obtain prescription data and other relevant medical data.

10. The method of claim 8, further comprising integrating with a payment gateway that allows patients to make payments for their purchases using the application itself.

11. The method of claim 8, further comprising employing a notification system to notify patients when their orders are ready for pickup or delivery.

12. A system for automatically summarizing a healthcare appointment between a healthcare provider and a patient, comprising:
a) audio input hardware for capturing audio from the appointment;
b) speech recognition software for processing the audio and generating text transcripts;
c) natural language processing (NLP) techniques for analyzing the text and identifying key topics and themes discussed in the appointment;
d) machine learning algorithms for identifying the most important parts of the appointment based on an organization's requirements;
e) a summary generation module for generating a summary of the appointment based on the results of the natural language processing and machine learning algorithms; and
f) a user interface for displaying the generated summary to the user, which includes features such as search, filter, and sharing.

13. The system of claim 12 further comprising an appointment management module for providing reminders for follow-up appointments and automatically scheduling appointments or providing recommendations or guidance to medical-related services.

14. The system of claim 12 further comprising a subscription generation module for automatically generating a prescription from the summary.

15. A system for assisting patients to purchase medication or rent medical equipment, comprising:
a) a processor configured to obtain real-time information about the availability of medical related products at nearby places of business, waiting time from queuing system associated with each place of business, and navigation data to each place of business;
b) a recommendation engine configured to combine the obtained information with the generated prescription (or other data from the summary) to provide a list of recommended places of business to purchase the medicines that appear in the prescription (or rent/buy medical equipment), wherein the recommendation engine further configured to sort the list of recommended places according to total time that it may take to complete the purchase, by considering the predicted waiting time, distance to the place of business, and other available navigation data; and
c) a dedicated application associated with the healthcare system, allowing the user to access the list of recommended places and complete the purchase in the most effective way, in terms of waiting time, distance, and other available navigation data.

16. The system of claim 15, further comprising:
a) an interface module configured to integrate with APIs provided by various third-party services to retrieve real-time data related to the availability of medical products, waiting times, and navigation data;
b) an interface module configured to integrate with the queuing system of each place of business to obtain waiting time data;
c) an interface module configured to integrate with a payment gateway that allows patients to make payments for their purchases using the application itself;
d) an interface module configured to integrate with geolocation services that identify the patient's location and provide navigation data to the recommended places of business; and
e) an interface module configured to integrate with electronic health record (EHR) systems to obtain prescription data and other relevant medical data.

17. The system of claim 16, further comprising a notification system configured to notify patients when their orders are ready for pickup or delivery.

18. The system of claim 15, further comprising a search/recommendation engine configured to receive a query for a desired product from the generated prescription and retrieve purchase-related data associated with the product from a plurality of components and services, wherein the purchase-related data includes availability, prices, distance, waiting times, and delivery options; a ranking algorithm configured to analyze the purchase-related data and generate a ranked list of search results based on a weighting of the purchase-related data, including predicted waiting times, geolocation with respect to the user's current location, and prices; and a user interface configured to display the ranked list of search results to the user, wherein the user can select a desired product from the search results and complete a purchase or rental transaction through the system.
